(19) United States
(12) Patent Application Publication
(10) Pub. No.: US 2025/0144171 A1
(43) Pub. Date: May 8, 2025

(54) METHODS AND COMPOSITIONS FOR TREATING DIABETES

(71) Applicant: TECHNION RESEARCH & DEVELOPMENT FOUNDATION LIMITED, Haifa (IL)

(72) Inventors: Shulamit LEVENBERG, Haifa (IL); Hagit SHOYHET, Haifa (IL); Dina SAFINA, Haifa (IL); Yifat HERMAN-BACHINSKY, Haifa (IL)

(21) Appl. No.: 18/837,691

(22) PCT Filed: Feb. 16, 2023
(86) PCT No.: PCT/IL2023/050166

§ 371 Date: Aug. 12, 2024

Related U.S. Application Data
(60) Provisional application No. 63/311,169, filed on Feb. 17, 2022.

(51) Int. Cl. A61K 38/17; A61K 9/50; A61P 3/10

(52) U.S. Cl. CPC A61K 38/177; A61K 9/5068; A61P 3/10

(57) ABSTRACT

The present invention is directed to, inter alia, therapeutic compositions comprising therapeutically effective amount of exosomes derived from cells having increased glucose transporter type 4 (GLUT4) activity, and a pharmaceutically acceptable. The invention is further directed to methods for reducing glucose levels in a subject in need thereof.

### CROSS-REFERENCE TO RELATED APPLICATIONS

This application claims the benefit of priority of U.S. Provisional Application No. 63/311,169, titled “METHODS AND COMPOSITIONS FOR TREATING DIABETES”, filed Feb. 17, 2022, the contents of which are incorporated herein by reference in their entirety.

### FIELD OF INVENTION

The present invention is in the field of exosomes for use of treatment of diabetes.

### BACKGROUND OF THE INVENTION

Diabetes mellitus is a group of metabolic diseases characterized by chronic hyperglycemia resulting from defects in insulin production, insulin secretion, insulin sensitivity, or a combination. The condition can be roughly divided to two types: type I diabetes—an autoimmune disease in which beta cells of the pancreas are destroyed by the immune system. Type II diabetes (DM2) is characterized mostly by insulin resistance in skeletal muscle and adipose tissues and thus elevated blood glucose levels. Maintaining steady and balanced blood glucose levels is crucial to sustaining healthy, normal life. Untreated glycemia (unbalanced blood-glucose levels) is hazardous to most organs leading to heart diseases, neural damage, muscle atrophy etc. according to CDC records, DM2 is the most common type of diabetes in the United States responsible for more than 90% of diabetes cases.

Up to this day, there are no treatments able to cure DM2, and most of the available therapy courses focus on balancing blood-glucose levels. In the early stages of the disease, this can be achieved by changing one's lifestyle, increasing physical activity, and altering the diet. However, in more progressive stages, the patients become increasingly dependent on insulin-stimulating drugs and eventually on insulin injections, indicating progressive insulin resistance and loss of function of pancreatic beta-cells.

The skeletal muscle tissue is one of the largest in the body and a central glucose consumer; therefore, skeletal muscle tissue plays a significant role in glucose homeostasis. A crucial component in skeletal muscle glucose uptake is the insulin-stimulated glucose transporter type 4 (GLUT4). The Glucose transporters are a family of transmembrane sugar transporters that mediate glucose uptake in eukaryotic cells. GLUT4 is the main insulin-stimulated glucose transporter, regulating glucose entry from the blood into adipose and muscle tissues. In response to insulin signaling, translocation of GLUT4 containing vesicles from the cytoplasm to the plasma membrane occurs, and the GLUT4 proteins are embedded in it in a transmembrane manner. Studies have demonstrated that GLUT4 density in muscle fibers from diabetic patients is reduced by 9% compared with the weight-matched obese subjects and by 18% compared to the lean control group. Thus, it has been proposed that a reduced slow-twitch fibers fraction, combined with a decrease in GLUT4 gene expression in slow-twitch fibers, is a central factor contributing to skeletal muscle insulin resistance in DM2. In vivo studies in GLUT4 transgenic and knockout animal models have provided insights into the pathogenesis of insulin resistance. Modified expression of GLUT4 in these models, either systemic or tissue-specific, affected whole-body insulin function as well as glucose metabolism.

Exosomes are a type of extracellular vesicles secreted by most eukaryotic cells and participate in intercellular communication. Exosomes vary in size but are usually in the range of 30-150 nm in diameter and contain proteins, DNA, mRNA, microRNA, long noncoding RNA, circular RNA, etc. Exosomes originate in the endocytic pathway. The exosomes biogenesis begins when the cytoplasmic membrane folds inwards to create an early secretory organelle called endosome; then intraluminal vesicles (ILVs) are created inside the endosome, termed a multi-vesicular body (MVBs). The late endosomes mature by acidification of their inner lumen, and eventually, the vesicles are released out of the cell as exosomes by fusion with the plasma membrane. In recent years, there has been a growing interest in using modified exosomes for therapeutic purposes, mainly as delivery systems. Since exosomes are derived from cells and naturally serve in cell-cell communication, they are not immunogenic and can reach a large number of cells. They can deliver mRNA, noncoding RNA, proteins, and small molecules such as anti-cancer drugs.

There is still a great need for specific exosomes being suitable and/or therapeutically improved, such as for use in the treatment of diabetes and/or metabolic syndrome.

### SUMMARY OF THE INVENTION

The present invention relates to exosomes secreted from cells having increased glucose transporter (e.g., GLUT4) levels, such as for method of restoring glucose homoeostasis and treatment of diabetes. The present invention further relates pharmaceutical compositions comprising exosomes from GLUT4 over-expressing cells for reducing elevated glucose levels, such as in subjects afflicted with diabetes and/or metabolic syndrome.

The present invention, in some embodiments, is based, at least in part, on the surprising findings that exosomes from comprising at least one protein in a modified amount compared to control exosomes, where found to increase glucose uptake in vitro, as well as improved response to glucose in murine diabetic model organism, compared to control exosomes. In some embodiments, the exosomes are derived from cells overexpressing GLUT4, such as, myogenic cells. Further, the therapeutic effects described herein, were shown to be even more pronounced when the myogenic exosomes were obtained from cells cultured on 3D scaffolds, compared to 2D.

According to one aspect, there is provided a pharmaceutical composition comprising a therapeutically effective amount of exosomes derived from cells having increased glucose transporter type 4 (GLUT4) activity.

According to another aspect, there is provided a pharmaceutical composition comprising exosomes comprising at least one protein listed under any one of Tables 1, 2, 3, and any combination thereof, wherein an amount of the at least one protein is modified in the exosomes compared to control exosomes.

According to another aspect, there is provided a method for treating or preventing diabetes mellitus or metabolic syndrome in a subject in need thereof, the method comprising the steps of administering a therapeutically effective amount of the pharmaceutical composition disclosed herein to the subject, thereby treating or preventing diabetes mellitus in the subject.

According to another aspect, there is provided a method for reducing glucose levels in a subject in need thereof, the method comprising the steps of administering a therapeutically effective amount of the pharmaceutical composition disclosed herein to the subject, thereby reducing glucose levels in the subject.

In some embodiments, the cells are selected from the group consisting of: (i) cells transduced or induced to increase GLUT4 gene expression; (ii) cells induced to increase GLUT4 membrane translocation; and (iii) cells having reduced GLUT4 degradation.

In some embodiments, the transduced cells are transduced by lentivirus comprising a nucleic acid sequence encoding GLUT4.

In some embodiments, the nucleic acid sequence encoding the GLUT4 is operably linked to a constitutive promoter.

In some embodiments, the cells are selected from the group consisting of: skeletal myocyte-derived cell, cardiomyocyte-derived cell, and adipocyte-derived cell.

In some embodiments, the cells are selected from the group consisting of: differentiated myotube, myocyte, and myoblast.

In some embodiments, the cells are differentiated myotube overexpressing GLUT4.

In some embodiments, the exosome are derived from cells cultured in a three-dimensional (3D) scaffold or in two dimensions (2D).

In some embodiments, the exosome are derived from cells cultured in a 3D scaffold.

In some embodiments, the exosomes comprise a glucose uptake molecule.

In some embodiments, the glucose uptake molecule is selected from a protein, DNA, mRNA, microRNA, long noncoding RNA, and circular RNA.

In some embodiments, the glucose uptake molecule is GLUT4.

In some embodiments, the exosomes comprise at least one protein listed under Tables 1, 2, 3, or any combination thereof, and wherein an amount of the at least one protein is modified in the exosomes compared to control exosomes.

In some embodiments, the at least one protein is listed under Tables 1, 3, or both, and the amount of the at least one protein is increased in the exosomes compared to the control exosomes.

In some embodiments, the at least one protein is listed under Table 2, and the amount of the at least one protein is decreased or reduced in the exosomes compared to the control exosomes.

In some embodiments, the control comprises exosomes derived from wild-type cells or exosomes derived from cells cultured in 2D.

In some embodiments, increased is by at least 1.5-fold compared to control exosomes.

In some embodiments, reduced amount is not more than 60% of control exosomes.

In some embodiments, the exosomes are derived from cells having increased GLUT4 activity.

In some embodiments, the pharmaceutical composition further comprises a pharmaceutically acceptable carrier.

In some embodiments, the pharmaceutical composition is for use in treatment or prevention of diabetes mellitus or metabolic syndrome in a subject in need thereof.

In some embodiments, the metabolic syndrome is selected from: obesity, pre-diabetes, and insulin resistance.

In some embodiments, the pharmaceutical composition is for use in reduction of glucose levels in a subject in need thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a micrographs showing a brightfield imaging of human myoblasts seeded in 2D (two dimensions).

FIGS. 2A-2B include fluorescent micrographs and a graph. (2A) Human myotubes cultured in 2D for 1 week stained for GLUT4 (green) DAPI for cell nuclei (blue) and Myogenin (magenta). 5× scale bar: 200=μm, 20× scale bar: 50=μm. (2B) GLUT4 fluorescence intensity quantification.

FIGS. 3A-3B include a graph and a micrograph showing myogenic exosomes characterization by (3A) nanoparticle tracking analysis (NTA) and (3B) cryo-TEM imaging. Scale bar=100 nm.

FIG. 4 includes a bar graph showing glucose uptake assay of two dimensional (2D) skeletal muscle tissue. From left to right: cells not incubated with exosomes (“no exosomes”); cells incubated with exosomes derived from wild-type (WT) skeletal muscle cells (“WT exosomes”); and cells incubated with exosomes derived from GLUT4 overexpressing skeletal muscle cells (“GLUT4 exosomes”).

FIGS. 5A-5C include bar graphs showing in vitro functionality of GLUT4 OE exosomes, as disclosed herein. (5A) 2-(N-(7-Nitrobenz-2-oxa-1,3-diazol-4-yl)Amino)-2-Deoxyglucose (2-NBDG) uptake assay on WT 3D muscle constructs incubated with PBS, WT 3D derived exosomes and overexpressed GLUT4 (OEG4) 3D derived exosomes for 4 days. (5B) 2-NBDG uptake assay repeated over the course of 5 days on WT 3D muscle constructs following incubation with WT-EMC or OEG4 3D derived exosomes. (5C) 2-NBDG uptake assay on WT 3D muscle constructs incubated with exosomes derived from MSCs and WT or OEG4 skeletal muscle cells cultured in 2D.

FIGS. 6A-6F include graphs showing in vivo functionality of GLUT4 OE exosomes, as disclosed herein. (6A-6B) Glucose tolerance test (GTT) analysis of diet induced obese (DIO) mice prior (6A) and after 3 days (6B) following three injections of 10⁹ OEG4-EMC and WT-EMC-derived exosomes. Injections were given with 3 days intervals. (6C) Area under the curve (AUC) statistical analysis of GTT measurements following exosomes injections (** p<0.01 ***p<0.001 ****p<0.0001, n=7). (6D) Area under the curve (AUC) statistical analysis of GTT measurements before and after injections of the group injected with saline (n=7). (6E) Area under the curve (AUC) statistical analysis of GTT measurements before and after injections of the group injected with WT-EMC derived exosomes (**** p<0.0001, n=7). (6F) Area under the curve (AUC) statistical analysis of GTT measurements before and after injections of the group injected with OEG4-EMC derived exosomes (* p<0.05 n=7).

FIGS. 7A-7C include graphs showing comparison between GLUT4 exosomes derived from 3D constructs vs. 2D differentiated cells. (7A) 2-NBDG uptake assay on WT 3D muscle constructs incubated with PBS, OEG4 3D derived exosomes and OEG4 2D derived exosomes for 4 days (n=3). (7B) GTT analysis of DIO mice prior and after 3 days following three injections of 109 OEG4-EMC and OEG4-2D derived exosomes. Injections were given with 3 days intervals. (7C) Area under the curve (AUC) statistical analysis of GTT measurements before and after injections of the group injected with OEG4-EMC and OEG4-2D derived exosomes (n=7).

### DETAILED DESCRIPTION OF THE INVENTION

### Pharmaceutical Composition

The present invention relates to a pharmaceutical composition comprising exosomes secreted from cells having increased glucose transporter levels, and use thereof, such as in method for restoring glucose homoeostasis (e.g., reducing hyperglycemia) and treatment of diabetes.

The present invention relates to a pharmaceutical composition comprising exosomes characterized by a particular protein abundance profile, and use thereof, such as in method for restoring glucose homoeostasis (e.g., reducing hyperglycemia) and treatment of diabetes.

According to one aspect, there is provided a pharmaceutical composition comprising a therapeutically effective amount of exosomes derived from cells having increased GLUT activity.

According to another aspect, there is provided a composition comprising an effective amount of exosomes derived from cells transduced or induced to increase GLUT (e.g., GLUT4) gene expression, activity, or both. In some embodiments, the cells are recombinant cells, gene edited cells, transgenic cells, or any combination thereof. In some embodiments, the composition further comprises a biologically acceptable carrier. In some embodiments, the carrier comprises a pharmaceutically acceptable carrier. In some embodiments, the composition is a pharmaceutical composition. In some embodiments, the pharmaceutical composition comprises a therapeutically effective amount of the exosomes being derived from cells being transduced or induced to increase GLUT (e.g., GLUT4) gene expression, activity, or both.

According to another aspect, there is provided a pharmaceutical composition comprising a therapeutically effective amount of exosomes derived from cells having increased GLUT content, and a pharmaceutically acceptable carrier.

According to another aspect, there is provided a composition comprising exosomes comprising at least one protein listed under any one of Tables 1, 2, 3, and any combination thereof, wherein an amount of said at least one protein is modified in said exosomes compared to control exosomes.

According to another aspect, there is provided a pharmaceutical composition comprising conditioned media derived from cells having increased GLUT content, and a pharmaceutically acceptable carrier. In some embodiment, the conditioned media comprises extracellular vesicles. In some embodiment, the conditioned media comprises exosomes.

In some embodiments, the pharmaceutical composition further comprises a pharmaceutically acceptable carrier.

In some embodiments, GLUT comprises or is: GLUT4, GLUT1, or both.

According to some embodiments, the cells form which exosomes or conditioned media comprising exosomes are extracted from are modulated to increase glucose transporter activity (e.g., increasing GLUT4 expression, translocation, or intrinsic activity).

According to some embodiments, the cells are transduced or induced to increase GLUT (e.g., GLUT4) gene expression. According to some embodiments, the cells are induced to increase GLUT (e.g., GLUT4) membrane translocation. According to some embodiments, the cells have reduced GLUT (e.g., GLUT4) degradation.

In some embodiments, the transduced cells are transduced by a lentivirus or a lentiviral vector comprising a nucleic acid sequence encoding GLUT4.

In some embodiments, the nucleic acid sequence encoding the GLUT4 is operably linked to a constitutive promoter.

Types of constitutive promoters are common and would be apparent to one of ordinary skill in the art. Non-limiting example of such constitutive promoter includes, but is not limited to, CMV promoter.

In one embodiment, a constitutive promoter comprises CMV promoter.

As used herein, “increased GLUT4 activity” includes, but not is limited to, increase of: GLUT4 gene expression, GLUT4 cellular content, membrane translocation by the cellular translocation machinery pathway, insulin signal transduction, glucose sensitivity in the absence or presence of insulin, or any combination thereof. In some embodiments, increased gene expression includes, but is not limited to, increased amount of the gene's mRNA molecules, increased amount of the translated polypeptides, or any combination thereof. In some embodiments, increased GLUT4 activity enhances cellular insulin sensitivity. In some embodiments, increased GLUT4 activity enhances cellular glucose uptake. In some embodiments, increased GLUT4 activity enhances cellular insulin sensitivity and cellular glucose uptake.

In some embodiments, insulin signal transduction inhibits GLUT4 degradation. In one embodiment, GLUT4 is degraded by a proteasome dependent pathway. In another embodiment, GLUT4 is degraded by an oxidative stress mediated pathway. Degradation of GLUT4 can be determined by any method known in the art, including, but not limited to, methods utilizing specific anti GLUT4 antibodies, comprising anti ubiquitinated-GLUT4 antibodies, among others. Non-limiting examples of methods which utilize antibodies include, but are not limited to, sandwich enzyme linked immunosorbent assay (ELISA, e.g., of either tissue homogenates, cell lysate or other biological fluids), 26S proteasome degradation assay, immunoprecipitation, immune-blotting, immune-histochemistry, immune-cytochemistry, any combination thereof, or any other method known to one of ordinary skill in the art.

In some embodiments, GLUT4 activity is increased by at least 2-fold, 5-fold, 10-fold, 25-fold or 100-fold, or any value and range therebetween. In another embodiment, GLUT4 activity is increased by 5-50%, 20-100%, 75-250%, 200-500%, 450-750%, or 600-1,000%. Each possibility represents a separate embodiment of the invention.

According to some embodiments, the exosomes comprise a glucose uptake molecule. According to some embodiments, the glucose uptake molecule is selected from a protein, DNA, mRNA, microRNA, long noncoding RNA, and circular RNA. According to some embodiments, the glucose uptake molecule is GLUT4. As used herein, the term “glucose uptake molecule” encompasses any molecule involved in, propagating, enhancing, increasing, promoting, any equivalent thereof, or any combination thereof, glucose uptake: into a cell (such as from culture medium, e.g., in vitro, ex vivo, or both), from the circulatory system (such as into a tissue or cell, e.g., in vivo), or both. In some embodiments, the glucose uptake molecule increases or promotes glucose uptake in vitro, in vivo, ex vivo, or any combination thereof. In some embodiments, the glucose uptake molecule increases or promotes glucose uptake in vitro. In some embodiments, the glucose uptake molecule increases or promotes glucose uptake in vivo. In some embodiments, the glucose uptake molecule increases or promotes glucose uptake ex vivo.

As used herein, the term “conditioned media” refers to media in which the cells of the invention (e.g., cells having increased GLUT4 activity or content) have been cultured, exosomes of the invention been secreted to, or both. In some embodiments, the cells have been cultured in the media for at least: 1 hour, 2 hours, 4 hours, 6 hours, 12 hours, 18 hours, 24 hours, 36 hours, 48 hours, 60 hours, 72 hours, or any value and range therebetween. Each possibility represents a separate embodiment of the invention. In some embodiments, the conditioned media comprises the exosomes of the invention. In some embodiments, the conditioned media comprises at least one protein secreted by cells having increased GLUT activity or content. In some embodiments, the conditioned media comprises the secretome of the cells. In some embodiments, the conditioned media comprises exosomes secreted by cells having increased GLUT activity or content, wherein the exosomes comprise at least one protein listed under any one of Tables 1-3, and having modified expression or abundance compared to control exosomes.

As used herein, the term “secretome”, refers to any substance(s) secreted by a cell. In some embodiments, a secretome comprises any or all of secreted proteins, secreted nucleic acid molecules, secreted vesicles, or any combination thereof.

As used herein, the term “extracellular vesicles” refers to all cell-secreted extracellular vesicles including but not limited to exosomes and micro-extracellular vesicles.

As used herein, the terms “exosome” or “exosomes” refer to cell-derived vesicle(s) of endocytic origin, with a size of 100-300 nm, and secreted from cells. In some embodiments, the exosomes are 150-200 nm in diameter. As demonstrated herein, the therapeutic exosomes of the invention has a size of 150-200 nm in diameter, which is a common size range for skeletal muscle exosomes.

In some embodiments, exosomes of the invention are myogenic exosomes, such as produced, secreted, or both, from myogenic cells or any muscle-related equivalent cell thereof.

The exosomes can be obtained by growing the cells in culture medium with serum depleted from exosomes or in serum-free media and subsequently isolating the exosomes by ultracentrifugation. Other methods associated with beads, columns, filters and antibodies may are also employed. In some embodiments, the exosomes are suspended in appropriate media for administration.

According to some embodiments, the cells are differentiated myotube overexpressing GLUT4. In some embodiments, GLUT4 overexpressing cells are cultured in or on a three-dimensional (3D) scaffold or in two dimensions (2D). In some embodiments, GLUT4 overexpressing cells are cultured in or on a 3D scaffold. In some embodiments, GLUT4 overexpressing cells are cultured in 2D. In some embodiments, exosomes derived from such cells (i.e., differentiated myotube having increased GLUT content or activity cultured in or on a 3D scaffold having increased GLUT content or activity) to resemble skeletal muscle tissue, are advantageous with regard to therapeutic activity contributing to glucose uptake. In some embodiments, exosomes derived from such cells (i.e., differentiated myotube having increased GLUT content or activity cultured in or on a 3D scaffold having increased GLUT content or activity) are advantageous with regard to therapeutic activity contributing to glucose uptake, as culturing thereof resembles skeletal muscle tissue.

In some embodiments, the exosome disclosed herein (e.g., extracellular vesicles) are derived from cells, such as myogenic cells, cultured in or on a 3D scaffold. In some embodiments, the 3D scaffold is an elastic 3D scaffold.

As used herein, the term “scaffold” refers to a structure that provides a surface suitable for adherence, attachment, anchoring, maturation, differentiation, proliferation, or any combination thereof, of cells. A scaffold may further provide mechanical stability and support. A scaffold may be in a particular shape or form so as to influence or delimit a three-dimensional shape or form assumed by a population of proliferating cells. As used herein three-dimensional shapes include: films, ribbons, cords, sheets, flat discs, cylinders, spheres, 3-dimensional amorphous shapes, or others.

In some embodiments, the scaffold is a porous matrix. In some embodiments, the porous scaffold comprises at least 50% porosity. In some embodiments, the porous scaffold comprises at least 60% porosity, at least 70% porosity, at least 75% porosity, at least 80% porosity, at least 85% porosity, at least 90% porosity, at least 92% porosity, or at least 95% porosity, and any value and range therebetween. In another embodiment, the porous scaffold comprises 45-55% porosity, 50-70% porosity, 60-80% porosity, 75-90% porosity, or 80-97% porosity. Each possibility represents a separate embodiment of the invention.

In another embodiment, the porous scaffold comprises pores having a diameter of at least 100 μm. In another embodiment, the porous scaffold comprises pores having a diameter of at least 120 μm. In another embodiment, the porous scaffold comprises pores having a diameter of at least 150 μm. In another embodiment, the porous scaffold comprises pores having a diameter of 100-900 μm. In another embodiment, the porous scaffold comprises pores having a diameter of 120-900 μm. In another embodiment, the porous scaffold comprises pores having a diameter of 120-850 μm. In another embodiment, the porous scaffold comprises pores having a diameter of 150-800 μm. In another embodiment, the porous scaffold comprises pores having a diameter of 200-800 μm. In another embodiment, the porous scaffold comprises pores having a diameter of 220-750 μm.

In another embodiment, the scaffold described herein comprises poly-l-lactic acid (PLLA). In another embodiment, the scaffold described herein comprises polylactic glycolic acid (PLGA). In another embodiment, the scaffold described herein comprises both poly-l-lactic acid (PLLA) and polylactic glycolic acid (PLGA). In another embodiment, the scaffold described herein comprises both poly-l-lactic acid (PLLA) and polylacticglycolicacid (PLGA). In another embodiment, PLLA and PLGA are in 1:3 to 3:1 w/w ratio. In another embodiment, PLLA and PLGA are in 1:2 to 2:1 w/w ratio. In another embodiment, PLLA and PLGA are in 1:1.5 to 1.5:1 w/w ratio. In another embodiment, PLLA and PLGA are in 1:1 w/w ratio.

In another embodiment, the porous scaffold is further coated with a polymer. In another embodiment, the porous scaffold is further coated with an extracellular matrix protein. In another embodiment, the porous scaffold is further coated with fibronectin. In another embodiment, the porous scaffold is further coated with polypyrrole. In another embodiment, the porous scaffold is further coated with polycaprolactone. In another embodiment, the porous scaffold is further coated with poly(ethersulfone). In another embodiment, the porous scaffold is further coated with poly(acrylonitrile-co-methylacrylate) (PAN-MA). In another embodiment, the porous scaffold further comprises a chemoattractant, such as, but not limited to laminin-1.

In another embodiment, a scaffold as described herein further comprises a material selected from: collagen-GAG, collagen, fibrin, PLA, PGA, PLA-PGA co-polymer, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, nonbiodegradable polyurethane, polyurea, poly(ethylene vinyl acetate), polypropylene, polymethacrylate, polyethylene, polycarbonate, poly(ethylene oxide), or any combination thereof.

In another embodiment, the porosity of the scaffold is controlled by a variety of techniques known to those skilled in the art. In another embodiment, as the porosity is increased, use of polymers having a higher modulus, addition of suffer polymers as a co-polymer or mixture, or an increase in the cross-link density of the polymer are used to increase the stability of the scaffold with respect to cellular contraction.

In another embodiment, the choice of polymer and the ratio of polymers in a co-polymer scaffold of the invention is adjusted to optimize the stiffness/porosity of the scaffold. In another embodiment, the molecular weight and cross-link density of the scaffold is regulated to control both the mechanical properties of the scaffold and the degradation rate (for degradable scaffolds). In another embodiment, the mechanical properties are optimized to mimic those of the tissue at the implant site. In another embodiment, the shape and size of the final scaffold are adapted for the implant site and tissue type. In another embodiment, scaffold materials comprise natural or synthetic organic polymers that can be gelled, or polymerized or solidified (e.g., by aggregation, coagulation, hydrophobic interactions, or cross-linking) into a hydrogel e.g., structure that entraps water and/or other molecules.

In another embodiment, scaffold materials comprise naturally occurring substances, such as, fibrinogen, fibrin, thrombin, chitosan, collagen, alginate, poly(N-isopropylacrylamide), hyaluronate, albumin, collagen, synthetic polyamino acids, prolamines, polysaccharides such as alginate, heparin, and other naturally occurring biodegradable polymers of sugar units. In another embodiment, structural scaffold materials are ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations.

In another embodiment, scaffolds as described herein are made by any of a variety of techniques known to those skilled in the art. Salt-leaching, porogens, solid-liquid phase separation (sometimes termed freeze-drying), and phase inversion fabrication are used, in some embodiments, to produce porous scaffolds.

In some embodiments, the extracellular vesicles (e.g., exosome) may be produced from an elastic three-dimensional (3D) scaffold as described in PCT/IL2021/050994 and Shaowei Guo, et al., Nano Letters, 2021 21 (6), 2497-2504, the contents of all of which are hereby incorporated by reference in their entirety.

In some embodiments, GLUT4 is human GLUT4. In some embodiments, the human GLUT4 comprises a polynucleotide sequence according to accession number M20747.1. In some embodiments, the human GLUT4 comprises a polypeptide sequence according to accession number AAA59189.1.

In some embodiments, GLUT is human GLUT1. In some embodiments, the human GLUT 1 comprises a polynucleotide sequence according to accession number NM_006516.3. In some embodiments, the human GLUT 1 comprises a polypeptide sequence according to accession number NP_006507.2.

According to some embodiments, the cells are selected from: differentiated myotube, myocyte, myoblast, or any combination thereof.

According to some embodiments, the cells are differentiated myotube overexpressing GLUT4.

In another embodiment, the cells express one or more markers selected from: desmin, myosin heavy chain (MYH), myogenin (MYOG), or any combination thereof.

None limiting examples for methods for detecting expression, presence, or both, of such markers are disclosed hereinbelow, and would be apparent to a skilled artisan.

In another embodiment, the cells are autologous cells. In another embodiment, the cells are allogeneic cells.

According to some embodiments, the cells are selected from: skeletal myocyte-derived cell, cardiomyocyte-derived cell, adipocyte-derived cell, or any combination thereof.

According to some embodiments, the cells may originate from any cell type capable of differentiating into a skeletal myocyte or a myotube. Non-limiting examples of such cells include, but are not limited to, mesenchymal stem cell (MSC), embryonic stem cell (ESC), adult stem cell, differentiated ESC, differentiated adult Stem cell, induced pluripotent Stem cell (iPSC), or any progenitor cell thereof.

One skilled in the art will appreciate that human embryonic pluripotent stem cells (hEPSCs) may be induced to differentiate into myogenic progenitor cells (iMPCs), by methods known in the art, such as described by Rao et al., (2018). Human induced pluripotent stem cells (iPS) may be induced to differentiate and form muscle fibers by methods known in the art, such as described by Chal et al., (2015). Human embryonic stem cells (hESCs) may be differentiated into skeletal myogenic cells as described by methods known in the art, such as by Shelton et al., (2014). Human mesenchymal stem cells (hMSCs) may be differentiated into skeletal myogenic cells by methods known in the art, such as described by Gang et al., (2004) or by Aboaloa and Han (2017). Human pluripotent stem cells (hPSCs) may be differentiated in vitro for generating muscle fibers and satellite-like cells methods known in the art, such as described by Chal et al., (2016).

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, the exosomes comprise at least one protein listed under Tables 1, 2, 3, or any combination thereof, wherein an amount of the at least one protein is modified in the exosomes compared to control exosomes.

In some embodiments, modified comprises altered. In some embodiments, modified comprises increased. In some embodiments, modified comprises reduced.

In some embodiments, the at least one protein is listed under Tables 1, 3, or both. In some embodiments, the amount of at least one protein listed under Tables 1, 3, or both is increased in the exosomes disclosed herein compared to control exosomes. In some embodiments, the amount of at least one protein listed under Tables 1, 3, or both is increased in the exosomes derived from myogenic cells overexpressing GLUT4 being cultured on 3D scaffold as disclosed herein, compared to control exosomes comprising exosomes derived from wild-type myogenic cell cultured on 3D scaffold. As used herein, the term “wild-type” encompasses any cell not overexpressing GLUT4.

In some embodiments, the at least one protein is listed under Table 2. In some embodiments, the amount of at least one protein is decreased in the exosomes disclosed herein compared to control exosomes. In some embodiments, the amount of at least one protein listed under Table 2 is decreased in the exosomes derived from myogenic cells overexpressing GLUT4 being cultured on 3D scaffold as disclosed herein, compared to control exosomes comprising exosomes derived from wild-type myogenic cell cultured on 3D scaffold.

In some embodiments, increase or increased amount comprises by at least: 1.5-fold increase, 1.5-fold increase, 1.7-fold increase, 2-fold increase, 2.5-fold increase, 5-fold increase, 6-fold increase, 7-fold increase, 9-fold increase, 10-fold increase, 20-fold increase, 50-fold increase, or 100-fold increase, compared to control exosomes, or any value and range therebetween. Each possibility represents a separate embodiment of the invention. In some embodiments, increase or increased amount comprises 1.5- to 100-fold increase, 1.5 to 10-fold increase, 1.7- to 8-fold increase, 2- to 15-fold increase, 2.5- to 8-fold increase, 5 to 25-fold increase, or 0.5- to 10-fold increase, compared to control exosomes, or any value and range therebetween. Each possibility represents a separate embodiment of the invention.

In some embodiments, reduced amount comprises not more than: 1%, 5%, 15%, 25%, 50%, 60%, 75%, 85%, 90%, 95%, 99%, of control exosomes, or any value and range therebetween. Each possibility represents a separate embodiment of the invention. In some embodiments, reduced amount comprises 1-99%, 5-99%, 10-99%, 20-99%, 50-99%, 70-99%, 80-99%, 10-70%, 20-65%, 40-70%, 45-80%, 50-70%, or 20-65%, of control exosomes. Each possibility represents a separate embodiment of the invention.

In some embodiments, control exosomes comprises exosomes derived from wild-type cells. In some embodiments, control exosomes comprises exosomes derived from wild-type cells cultured on 3D scaffolds, in 2D, or both. In some embodiments, control comprises exosomes derived from myogenic cells cultured in 2D. In some embodiments, control exosomes comprises exosomes derived from non-myogenic cells. In some embodiments, control exosomes comprises exosomes derived from myogenic cells not being cultured on 3D scaffold. In some embodiments, control comprises exosomes derived from myogenic cells overexpressing GLUT4 being cultured in 2D.

In some embodiments, the pharmaceutical composition further comprises a pharmaceutically acceptable carrier.

In some embodiments, the pharmaceutical composition is for use in reducing glucose levels in a subject in need thereof.

In some embodiments, the pharmaceutical composition is for use in treatment or prevention of diabetes mellitus or metabolic syndrome in a subject in need thereof.

In some embodiments, a metabolic syndrome is selected from: obesity, pre-diabetes insulin resistance, any symptom associated therewith, or any combination thereof.

As used herein, the term “carrier”, “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Use

According to another aspect, there is provided a method for reducing glucose levels in a subject in need thereof, the method comprising administering a therapeutically effective amount of exosomes as disclosed herein to the subject.

According to another aspect, there is provided a method for reducing glucose levels in a subject in need thereof, the method comprising administering a pharmaceutical composition as disclosed herein to the subject.

According to another aspect, there is provided a method for treating or preventing diabetes mellitus or metabolic syndrome in a subject in need thereof, the method comprising administering a therapeutically effective amount of exosomes as disclosed herein to the subject.

According to another aspect, there is provided a method for treating or preventing diabetes mellitus or metabolic syndrome in a subject in need thereof, the method comprising administering a pharmaceutical composition as disclosed herein to the subject.

The present invention features compositions, and methods for treating, preventing, and reducing metabolic disorders. This invention is particularly useful for treating patients having or at risk of having any condition that is characterized by a state of hyperglycemia, which may be caused, for example, by an alteration in the insulin signaling pathway (e.g., a reduction in insulin production, resistance to insulin, or both).

As used herein, the term a “metabolic syndrome, disease, disorder, or condition” refers to any disease or disorder characterized by excess abdominal fat, hypertension, abnormal fasting plasma glucose level or insulin resistance, high triglyceride levels, low high-density lipoprotein (HDL) cholesterol level, and any combination thereof. In some embodiments, the metabolic syndrome disorders which can be treated according to the present invention are diverse and will be easily understood by the skilled artisan. Without any limitation mentioned are obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance or insulin-resistance related. High risks of metabolic syndrome disease include, but are not limited to, obstructive sleep apnea, nonalcoholic steatohepatitis, chronic kidney disease, polycystic ovary syndrome and low plasma testosterone, erectile dysfunction, or both.

By “treating, reducing, treatment, preventing, prevention of a metabolic disorder” it is meant ameliorating such a condition before or after it has occurred, or at relieving at least one symptom associated therewith. As compared with an equivalent untreated control, such reduction or degree of prevention is at least 5%, 10%, 20%, 40%, 50%, 60%, 80%, 90%, 95%, or 100% as measured by any standard technique.

By “a metabolic disorder” it is meant any pathological condition resulting from an alteration in a patient's metabolism. Such disorders include those resulting from an alteration in glucose homeostasis resulting, for example, in hyperglycemia. According to this invention, an alteration in glucose levels is typically an increase in glucose levels by at least 5%, 10%, 20%, 30%, 40%, 50%, 60%, 70%, 80%, 90%, or even 100% relative to such levels in a healthy individual. Metabolic disorders include obesity and diabetes (e.g., diabetes type I, diabetes type II, MODY, and gestational diabetes), satiety, and endocrine deficiencies of aging.

As used herein, the terms “treat,” “treating,” “treatment,” and the like refer to reducing or ameliorating a disease or condition, e.g., diabetes, hyperglycemia, insulin resistance, and/or symptoms associated therewith. In some embodiments, treatment includes the partial or complete regeneration of normoglycemia in a subject. It will be appreciated that, although not precluded, treating a disease or condition does not require that the disease, condition, or symptoms associated therewith be completely eliminated.

The term “diabetes” refers to the metabolic disease diabetes mellitus. In some embodiments, this refers to type I diabetes, also known as insulin-dependent diabetes mellitus. In other embodiments, this refers to type II diabetes, also known as adult-onset diabetes mellitus.

By “reducing glucose levels” is meant reducing the level of glucose by at least 10%, 20%, 30%, 40%, 50%, 60%, 70%, 80%, 90%, 95%, or 100% relative to an untreated control. Desirably, glucose levels are reduced to normoglycemic levels.

As used herein, the term “normoglycemic levels” or “normoglycemia” refers to the normal levels of glucose in the blood of healthy people. In some embodiments, normoglycemia is glucose levels of about 70 to 100 milligrams per deciliter (mg/dL) of blood in healthy people pre-meal (e.g., fasting). In some embodiments, normoglycemia is glucose levels of about 75 to 100 mg/dL of blood in healthy people pre-meal (e.g., fasting). In some embodiments, normoglycemia is glucose levels of about 85 to 100 mg/dL of blood in healthy people pre-meal (e.g., fasting). In some embodiments, normoglycemia is glucose levels of about 70 to 95 mg/dL of blood in healthy people pre-meal (e.g., fasting). In some embodiments, normoglycemia is glucose levels of about 75 to 90 mg/dL of blood in healthy people pre-meal (e.g., fasting).

In some embodiments, normoglycemia is glucose levels up to 140 mg/dL of blood in healthy people postprandial (e.g., 2 hours after eating). In some embodiments, normoglycemia is glucose levels of about 80 to 140 mg/dL of blood in healthy people postprandial (e.g., after eating). In some embodiments, normoglycemia is glucose levels of about 90 to 140 mg/dL of blood in healthy people postprandial (e.g., after eating). In some embodiments, normoglycemia is glucose levels of about 100 to 140 mg/dL of blood in healthy people postprandial (e.g., after eating). In some embodiments, normoglycemia is glucose levels of about 110 to 140 mg/dL of blood in healthy people postprandial (e.g., after eating). In some embodiments, normoglycemia is glucose levels of about 120 to 140 mg/dL of blood in healthy people postprandial (e.g., after eating). In some embodiments, normoglycemia is glucose levels of about 130 to 140 mg/dL of blood in healthy people postprandial (e.g., after eating). In some embodiments, normoglycemia is glucose levels less or equal to 140 mg/dL of blood in healthy people postprandial (e.g., 2 hours after eating).

As used herein, the term “impaired fasting glucose (IFG)” refers to fasting glucose levels between 100-125 mg/dL.

In another embodiment, a glucose level lower than the mentioned herein normoglycemia is hypoglycemia. In another embodiment, a glucose level greater than the mentioned herein normoglycemia is considered hyperglycemia. In another embodiment, a subject having fasting blood glucose level of 100-125 mg/dL or 2 hours postprandial (e.g. test by glucose tolerance test) level of 140-199 mg/dL, is considered pre-diabetic and/or having insulin resistance.

In some embodiments, hyperglycemia of 200 mg/dL and above, without returning to basal levels within a period of 2 hours after a glucose tolerance test of 75 gr, is indicative of diabetes.

In some embodiments, glucose levels are measured by means of a blood test after fasting (e.g., FGT). In some embodiments, glucose levels are measured by means of an oral glucose tolerance test (e.g., OGTT). In some embodiments, glucose levels are estimated by the level of glycosylated hemoglobin (e.g., HbA1C). As apparent to one skilled in the art, normoglycemia is having up to 5.7% HbA1C. In another embodiment, hyperglycemia is having HbA1C level of 6.5% or more. In another embodiment, HbA1C level of 5.7-6.4% is indicative of prediabetes. In another embodiment, HbA1C level of 6.5% or more is indicative of diabetes.

In some embodiments, a “therapeutically effective amount of exosomes” is sufficient for maintaining glucose homeostasis at levels of less than or equal to 100 mg/dL at fasting. In another embodiment, the fi therapeutically effective amount of exosomes is sufficient for maintaining glucose homeostasis at levels of less than or equal to 110, 120 or 130 mg/dL at fasting. In another embodiment, fasting is for at least 1, 4, 8, 12 or 14 hours, and any range and value therebetween. In another embodiment, fasting is for 1-3 h, 2-5 h, 3-8 h, 4-6 h, 4-9 h, 7-12 h, 8-16 h, 14-20 h, 12-24 h. Each possibility represents a separate embodiment of the invention.

In some embodiments, the therapeutically effective amount of exosomes of the present invention is sufficient for maintaining glucose homeostasis at levels of less than 140 mg/dL postprandial. In another embodiment, the therapeutically effective amount of exosomes is sufficient for maintaining glucose homeostasis at levels of less than 150 or 160 mg/dL postprandial. In another embodiment, postprandial is not more than 15, 30, 45 or 60 min postprandial, and any value and range therebetween. In another embodiment, postprandial is not more than 2, 3, 4, 5 or 6 hours postprandial, and any value and range therebetween. In another embodiment, postprandial is 0.5-1.5 h, 1-3 h, 2-4 h, 3-5 h, or 3-7 h postprandial. Each possibility represents a separate embodiment of the invention.

As used herein, the cell described herein is a recombinant cell. In some embodiments, the term “recombinant cell” as used herein, refers to a cell whose genetic composition was modified. In one embodiment, a recombinant cell comprises exogenous polynucleotide. In another embodiment, a recombinant cell expresses an exogenous polynucleotide. In one embodiment, a recombinant cell constitutively expresses an endogenous polynucleotide. In another embodiment, a recombinant cell conditionally expresses an endogenous polynucleotide. A non-limiting example of constitutive expression is achieved by contacting a cell with an endogenous polynucleotide operably linked to a constantly operating promoter polynucleotide. In another embodiment, recombinant cell facultatively expresses endogenous or exogenous polynucleotide in response to a specific stimulation (e.g., induced or conditional expression). In another embodiment, recombinant cell expresses endogenous or exogenous polynucleotide indefinitely. Recombinant expressions systems are well known to one skilled in the art, non-limiting examples of which include the Tetracycline-controlled transcriptional activation (“Tet-on/Tet off”), Actin-GAL4-UAS, IPTG-inducible conditional expression, or others.

In another embodiment, the expression of the GLUT4 gene in the recombinant cell is upregulated by at least 2-fold, 3-fold, 4-fold, 5-fold, 6-fold, 8-fold or 10-fold, and any value and range therebetween. In another embodiment, the expression of the GLUT4 gene in the recombinant cell is upregulated by 5-50%, 40-100%, 75-250%, 200-350%, 300-500%, 400-750%, or 700-1,500%. Each possibility represents a separate embodiment of the invention.

In another embodiment, increased GLUT4 activity is in the level sufficient to restore glucose homeostasis. In another embodiment, increased GLUT4 activity is in the level sufficient to maintain glucose homeostasis. In another embodiment, increased GLUT4 activity is in the level sufficient to rectify glucose homeostasis. In another embodiment, increased GLUT4 activity is in the level sufficient to reduce hyperglycemia in a subject. In another embodiment, increased GLUT4 activity is in the level sufficient to restore normoglycemia in a subject afflicted with hyperglycemia. In another embodiment, increased GLUT4 activity is in the level sufficient to reduce fasting glucose levels to levels less than or equal to 80 mg/dL in a subject. In another embodiment, increased GLUT4 activity is in the level sufficient to reduce fasting glucose levels to levels less than or equal to 90 mg/dL in a subject. In another embodiment, increased GLUT4 activity is in the level sufficient to reduce fasting glucose levels to levels less than or equal to 95 mg/dL in a subject. In another embodiment, increased GLUT4 activity is in the level sufficient to reduce fasting glucose levels to levels less than or equal to 100 mg/dL in a subject. In another embodiment, increased GLUT4 activity is in the level sufficient to reduce fasting glucose levels to levels less than or equal to 105 mg/dL in a subject. In another embodiment, increased GLUT4 activity is in the level sufficient to reduce fasting glucose levels to levels less than or equal to 110 mg/dL in a subject. In another embodiment, increased GLUT4 activity is in the level sufficient to reduce fasting glucose levels to levels less than or equal to 115 mg/dL in a subject. In another embodiment, increased GLUT4 activity is in the level sufficient to reduce fasting glucose levels to levels less than or equal to 120 mg/dL in a subject. In another embodiment, increased GLUT4 activity is in the level sufficient to reduce postprandial glucose levels to levels less than 135 mg/dL in a subject. In another embodiment, increased GLUT4 activity is in the level sufficient to reduce postprandial glucose levels to levels less than 140 mg/dL in a subject. In another embodiment, increased GLUT4 activity is in the level sufficient to reduce postprandial glucose levels to levels less than 145 mg/dL in a subject. In another embodiment, increased GLUT4 activity is in the level sufficient to reduce postprandial glucose levels to levels less than 150 mg/dL in a subject. In another embodiment, increased GLUT4 activity is in the level sufficient to reduce postprandial glucose levels to levels less than 155 mg/dL in a subject. In another embodiment, increased GLUT4 activity is in the level sufficient to reduce postprandial glucose levels to levels less than 160 mg/dL in a subject.

In some embodiments, a recombinant cell of the present invention comprises a cell in which GLUT4 levels have been directly elevated. As used herein, the term “directly elevated levels of GLUT4” refers to contacting a cell with a polynucleotide comprising a GLUT4 encoding sequence and inducing its expression, thereby resulting in its elevated levels in the cell. In some embodiments, the elevated levels are increased levels of the GLUT4 encoding gene transcription. In some embodiments, the elevated levels are increased amounts of the GLUT4 mRNA molecules. In some embodiments, the elevated levels are increased rates of the GLUT4 mRNA translation. In some embodiments, the elevated levels are increased GLUT4 mRNA stability. In some embodiments, the elevated levels are increased amounts of the GLUT4 polypeptide. In some embodiments, the elevated levels are achieved by a vector or a plasmid transfection. In some embodiments, the vector or plasmid is transfected to a cell of the invention. In some embodiments, the vector comprises a polynucleotide comprising GLUT4 encoding sequence. In some embodiments, the increased levels of the GLUT4 encoding gene are induced by GLUT4 gene editing. In some embodiments, the gene editing comprises molecular alterations in the GLUT4 genomic polynucleotide's sequence which induce or promotes the gene's over expression. In some embodiments, the gene editing is achieved by Clustered Regularly Interspaced Short Palindromic Repeats (CRISPR) system.

In some embodiments, a recombinant cell of the invention comprises a cell in which GLUT4 levels have been indirectly elevated. As used herein, the term “indirectly elevated levels of GLUT4” refers to blocking negative regulators inhibiting GLUT4 activity, thereby resulting in its elevated activity. In some embodiments, a negative regulator is a transcription inhibitor. In some embodiments, a negative regulator is a translation inhibitor.

In some embodiments, a negative regulator inhibits GLUT4 migration via the secretory pathway. In some embodiments, a negative regulator inhibits trafficking of the GLUT4 polypeptide to the cellular membrane. In some embodiments, a negative regulator is an antibody. In some embodiments, a negative regulator is an RNA molecule. In some embodiments, a negative regulator is an antisense RNA molecule. In some embodiments, a negative regulator is a micro RNA molecule (miRNA). In some embodiments, a negative regulator is a protease inhibitor. In some embodiments, a negative regulator is steroid.

The term “polynucleotide” refers to a nucleic acid (e.g., DNA or RNA) sequence that comprises coding sequences necessary for the production of a polypeptide. In one embodiment, a polynucleotide refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In one embodiment, “complementary polynucleotide sequence” refers to a sequence, which results from reverse transcription of messenger RNA using a reverse transcriptase or any other RNA dependent DNA polymerase. In one embodiment, the sequence can be subsequently amplified in vivo or in vitro using a DNA polymerase.

In one embodiment, “genomic polynucleotide sequence” refers to a sequence derived (isolated) from a chromosome and thus it represents a contiguous portion of a chromosome.

In some embodiments, the molecule blocking a negative regulator inhibiting GLUT4 activity is a monoclonal antibody. In some embodiments, the molecule blocking a negative regulator inhibiting GLUT4 activity is a recombinant monoclonal antibody. In some embodiments, the molecule blocking a negative regulator inhibiting GLUT4 activity is a polyclonal antibody. In some embodiments, the molecule blocking a negative regulator inhibiting GLUT4 activity is a recombinant polyclonal antibody.

According to the method disclosed herein, in some embodiments thereof, the molecule blocking a negative regulator inhibiting GLUT4 activity is a nucleic acid. In some embodiments, the molecule blocking a negative regulator inhibiting GLUT4 activity has one or more chemical modifications to the backbone or side chains as described herein. In some embodiments, the molecule blocking a negative regulator inhibiting GLUT4 activity is a RNA interfering (RNAi) molecule. In some embodiments, the interfering RNA is a small hairpin RNA (shRNA), a small interfering RNA (siRNA), a double stranded RNA (dsRNA), or a miRNA antagonizing RNA (antagomiR). According to the method disclosed herein, in some embodiments thereof, blocking a negative regulator inhibiting GLUT4 expression and/or activity is by means of the CRISPR Cas system.

Inhibitory nucleic acids useful in the present methods and compositions, in some embodiments thereof, include antisense oligonucleotides, ribozymes, external guide sequence (EGS) oligonucleotides, siRNA compounds, single- or double-stranded RNAi compounds such as siRNA compounds, modified bases/locked nucleic acids (LNAs), antagomirs, peptide nucleic acids (PNAs), or other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, siRNA; a micro RNA (miRNA); a small temporal RNA (stRNA); shRNA; small RNA-induced gene activation (RNAa); small activating RNAs (saRNAs), or combinations thereof.

As used herein, the term “an interfering RNA” refers to any double stranded or single stranded RNA sequence, capable-either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long.

As would be apparent to one of ordinary skill in the art, a CRISPR Cas system as can be used according to the disclosed method, utilizes a CRISPR complex binding to a polynucleotide target, such that the binding results in increased or decreased expression of the polynucleotide. In some embodiments, the method further comprises delivering one or more vectors to the cells of the invention, wherein the one or more vectors drive expression of one or more of: the CRISPR enzyme, the guide sequence linked to the tracer mate sequence, or the tracer sequence.

The inhibitory nucleic acids useful according to the herein disclosed method have at least 80% sequence complementarity to a target region within the target nucleic acid, e.g., 90%, 95%, or 100% sequence complementarity to the target region within the targeted gene, and any value and range therebetween. Each possibility represents a separate embodiment of the invention.

In some embodiments, the molecule blocking a negative regulator inhibiting GLUT4 activity is a peptide mimetic or peptidomimetic. The terms “peptide mimetics” or “peptidomimetics” as used herein, refer to structures which serve as substitutes for peptides in interactions between molecules (Morgan et al., 1989). Peptide mimetics include synthetic structures which may or may not contain amino acids and/or peptide bonds but retain the structural and functional features of a peptide, or agonist or antagonist (i.e. enhancer or inhibitor) of the invention. Peptide mimetics also include peptoids, oligopeptoids (Simon et al., 1972); and peptide libraries containing peptides of a designed length representing all possible sequences of amino acids corresponding to a motif, peptide, or agonist or antagonist (i.e. enhancer or inhibitor) of the invention.

In one embodiment, the present invention provides a vector or a plasmid comprising the nucleic acid molecule as described herein. In one embodiment, a vector or a plasmid is a composite vector or plasmid. In one embodiment, a vector or a plasmid is a man-made vector or plasmid comprising at least one DNA sequence which is artificial. In one embodiment, the present invention provides a vector or a plasmid comparing: pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In one embodiment, the present invention provides a vector or a plasmid comprising regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Typically, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

The term “subject” or “patient” refers to an animal which is the object of treatment, observation, or experiment. By way of example only, a subject includes, but is not limited to, a mammal, including, but not limited to, a human or a non-human mammal. Non-limiting examples of a non-human mammal include, primate, murine, bovine, equine, canine, ovine, or feline subject.

It should be noted that the exosomes can be administered as the pharmaceutical composition and can be administered alone or as an active ingredient in combination with pharmaceutically acceptable carriers, diluents, adjuvants, and vehicles. The composition can also be administered orally, subcutaneously, or parenterally including intravenous, intraarterial, intramuscular, intraperitoneally, intratonsillar, and intranasal administration as well as intrathecal and infusion techniques. The patient being treated is a warm-blooded animal and, in particular, mammals including humans. The pharmaceutically acceptable carriers, diluents, adjuvants, and vehicles generally refer to inert, non-toxic solid or liquid fillers, diluents or encapsulating material not reacting with the active ingredients of the invention.

The doses can be single doses or multiple doses over a period of several days, weeks, months or even years. The treatment generally has a length proportional to the length of the disease process and drug effectiveness and the patient species being treated.

As used herein, the terms “carrier”, “adjuvant” or “excipient” refer to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents that may be useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

The carrier may comprise, in total, from about 0.1% to about 99.99999% by weight of the pharmaceutical compositions presented herein.

As used herein, the terms “therapeutically active molecule” or “therapeutic agent” mean a molecule, group of molecules, complex or substance administered to an organism for diagnostic, therapeutic, preventative medical, or veterinary purposes. This term includes pharmaceuticals, e.g., small molecules, treatments, remedies, biologics, devices, and diagnostics, including preparations useful in clinical screening, prevention, prophylaxis, healing, imaging, therapy, surgery, monitoring, and the like. This term can also specifically include nucleic acids and compounds comprising nucleic acids that produce a bioactive effect, for example.

The term “therapeutically effective amount” refers to the concentration of exosomes derived (e.g., secreted) from cells that over-express GLUT4 and are normalized to body weight (BW) that is effective to treat a disease or disorder in a mammal. The term “a therapeutically effective amount” refers to an amount effective, at dosages and for periods of time necessary, to achieve the desired therapeutic or prophylactic result. A physician or veterinarian of ordinary skill can readily determine and prescribe the effective amount of the bioactive agent required.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention, in some embodiments, include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cell Culture

### Human Myoblast

Human skeletal muscle cells (hSkMC) were cultured in hSkMC medium (ScienCell) supplemented with 10% of fetal bovine serum (FBS), 1% Pen Strep antibiotic (PS) and skeletal muscle growth factors, all provided in the medium kit. GLUT4 overexpression in human myoblasts

GLUT4 overexpression in human myoblasts was achieved via a lentiviral transduction. The plasmid contained the human gene for GLUT4 as well as a reporter gene-mCherry, under a CMV promoter. The cells were selected for puromycin resistance and validated by following the reporter expression.

### Glucose Uptake in Myotubes

Differentiated cells were washed twice with PBSx1 and KRP buffer was added to each well for serum starvation and incubated 37° C. 5% CO₂ for 3 hours.

After starvation, Insulin was added to the cells to a final concentration of 100 mM and incubated 37° C. 5% CO2 for 20 minutes.

2-NBDG to a final concentration of 120 μM was added to the relevant wells and incubated 37° C., 5% CO₂ for 20 minutes.

The reaction was quickly halted by washing thrice with cold PBSx1 and fluorescence was measured: Excitation WL-465 nm, Emission WL-540 nm.

### Exosomes Isolation

Exosomes were isolated from 7 days differentiated myotubes. 48 hours prior to isolation cells were transferred to exosome-depleted differentiation medium (exosome-depleted FBS was prepared according to manufacturer's instructions using Norgen Biotek FBS Exosome Depletion Kit).

On the day of the isolation, conditioned medium was collected and centrifuged 5 minutes 500×g 4° C., the supernatant was decanted and transferred to a clean tube. Then it was centrifuged 30 minutes 2,000×g 4° C. the supernatant was decanted and transferred to a clean tube. A 30% PEG solution was added 1:1 to the medium, inverted a few times to mix it and incubated 4° C. O.N. The mixture was then centrifuged 60 minutes 10,000×g 4° C., the supernatant was removed and allowed to drain on a clean paper towel for 5 min, tapping from time to time to remove polyethylene glycol (PEG). The final pellet was resuspended in 200 μl sterile and filtered PBS×1.

### Example 1

### Human Skeletal Muscle Tissue Characterization in 2D

Human skeletal muscle cells were engineered by lentiviral infection of human primary myoblasts with GLUT4 containing plasmid under CMV overexpression promoter. Two lines of human GLUT4 over expressing (G40E) cells were created, one contained mCherry reporter downstream to GLUT4 and the other one solely included GLUT4. The mCherry is an RFP-like reporter which is helpful in real-time assessment of the transduction efficiency.

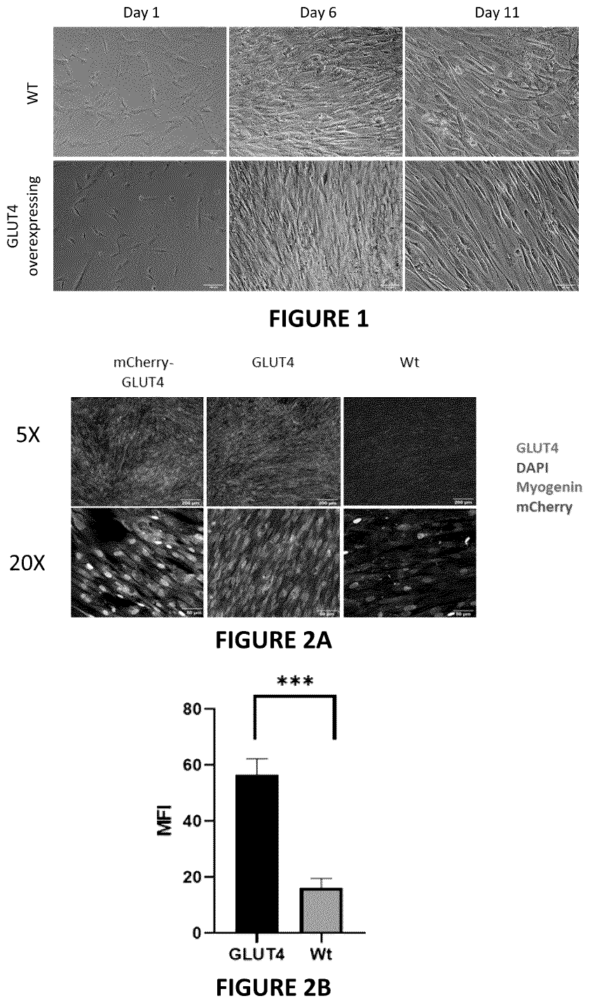

First, human myoblasts, both wildtype and G40E were seeded in a 24 well-plate 5×10⁴ cells/well and cultured for 7 days. The cells were seeded in commercially hSkMC growth medium and after 24 hours were transferred to differentiation medium based on DMEM supplemented with 5% horse-donor serum. As shown in FIG. 1, after as little as 6 days the vast majority of cells were fused and myotubes were created.

Additionally, the inventors quantified and compared GLUT4 in wt and G40E cells. For this, the transduced cells were selected, seeded in a 24 well-plate at a density of 5×10⁴ cells/well and differentiated in 2D for 7 days, and immunostained for GLUT4 (green), DAPI (blue) and myogenin (magenta). All images were taken under the same conditions and processed similarly. The 20× magnification shows a bright myogenin signal, indicating the cells are committed to differentiation, which can also be assessed by their elongated morphology (FIG. 2A). As can be noticed in FIGS. 2A-2B, a significant difference was measured in GLUT4 fluorescence intensity between G40E and wt cells, indicating the transduction was successful.

### Example 2

### Skeletal Muscle Exosomes Isolation and Characterization

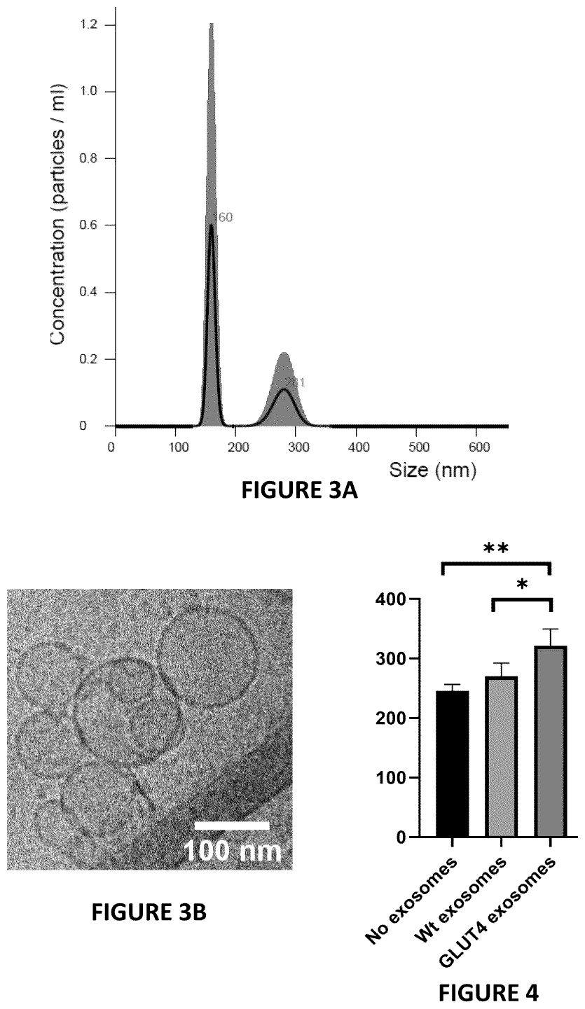

Myogenic exosomes were isolated from the conditioned medium of differentiated L6 myotubes after 7 days differentiation in 2D using PEG precipitation. The isolated exosomes were analyzed using NTA nanosight as presented in FIG. 3A. As can be seen, the majority of particles are 150-200 nm in diameter which is supported by the literature as a common size range for skeletal muscle exosomes. Comparison of exosomes derived from GLUT4 OE cells vs WT derived exosomes showed no significant difference in size and concentration parameters. Exosomes were also imaged by cryo-transmission electron microscopy (cryo-TEM; FIG. 3B).

### Example 3

### GLUT4 OE Myotubes Derived Exosomes Increase Glucose Uptake in Wt Myotubes

Next, the inventors have examined whether GLUT4 OE derived exosomes have an activity related to diabetes and/or metabolic syndrome. For this, a glucose uptake assay was performed, using the fluorescent analogue 2-NBDG. Wild type (WT) cells were incubated with WT and GLUT4 OE derived exosomes for 4 days prior to the experiment, and their glucose uptake in the presence of insulin was measured. The preliminary glucose uptake results indicate that GLUT4 derived exosomes increase glucose uptake in WT cells at about 40% compared to cells that were not incubated with exosomes at all, and at about 20% compared to cells that were incubated with wt derived exosomes (FIG. 4).

All in all, according to the current results, it is suggested that: (i) myogenic exosomes contain either mRNA of GLUT4 or of other components in the glucose uptake pathway, may lead to improved activity of the pathway following incubation (e.g., in the context of glucose uptake, insulin responsiveness, etc.); and (ii) administration of myogenic exosomes purified from GLUT4 overexpressing tissue (e.g., by injection), may reduce blood glucose levels in diabetic subjects, e.g., mice, as exemplified herein.

### Example 4

### In Vitro Functionality of GLUT4 OE Exosomes

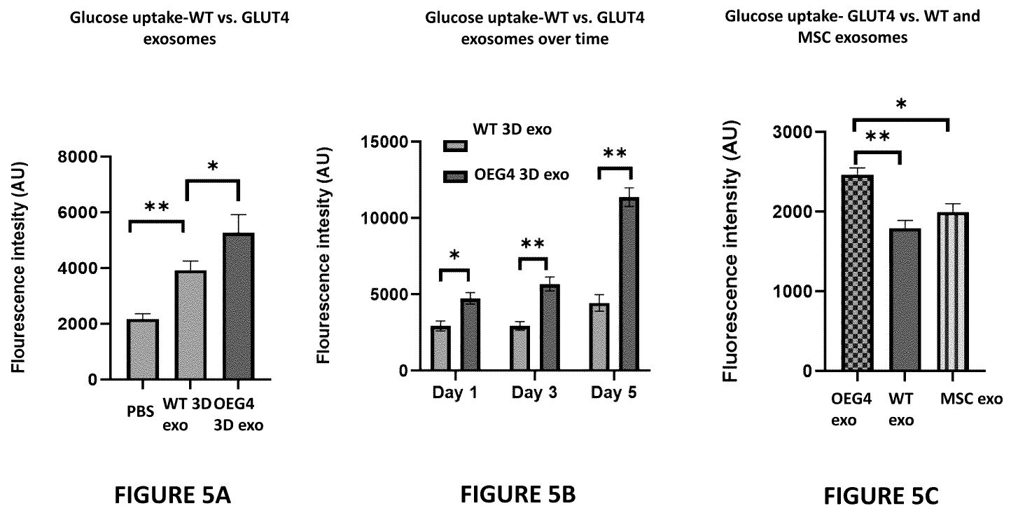

To assess function of myogenic exosomes in vitro, the inventors used a glucose-analogue (2-NBDG) uptake assay. WT and GLUT4 OE exosomes isolated from 3D muscle constructs seeded with WT and OEG4 human SkMC, respectively, were incubated for 4 days with WT hSkMC constructs differentiated for 3 weeks. The results show an increase in glucose uptake capacity of muscle constructs incubated with OEG4 3D exosomes compared to WT 3D exosomes and compared to PBS control (FIG. 5A). WT 3D exosomes also increased glucose uptake compared to PBS control. The inventors also examined the potential long-term effect of the system. For that, the inventors performed a glucose-uptake assay for 5 days. After incubation with the exosome, the latter were washed, and culture medium was replaced with clean/fresh medium. The results show an enhanced effect on glucose uptake over time, suggesting involvement of regulatory mechanisms in (FIG. 5B). In order to delineate the unique activity of myogenic exosomes compared to other types of exosomes, e.g., being derived from other types of cells, the inventors isolated exosomes from 2D cultures of human endothelial cells (HUVECs), and mesenchymal stem cells (MSCs). The exosomes derived from these cells were compared to exosomes derived from OEG4 2D hSkMC. The results show an enhanced effect of myogenic exosomes compared to exosomes from other cells, in the context of glucose uptake (FIG. 5C), thus, suggesting that myogenic exosomes harbor metabolic regulation activity(s), and are therefore of therapeutic relevancy for diabetes and/or metabolic syndrome, or symptoms associated therewith.

### Example 5

### In Vivo Functionality of GLUT4 OE Exosomes

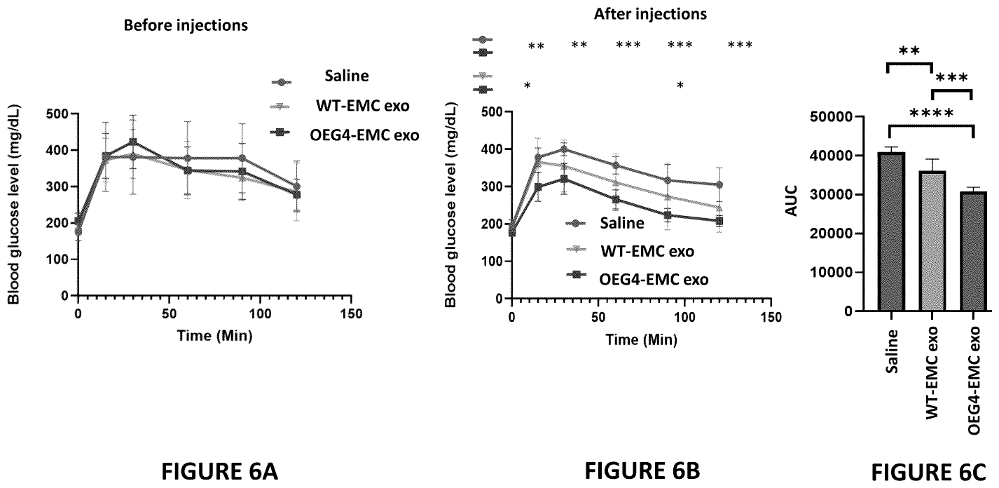

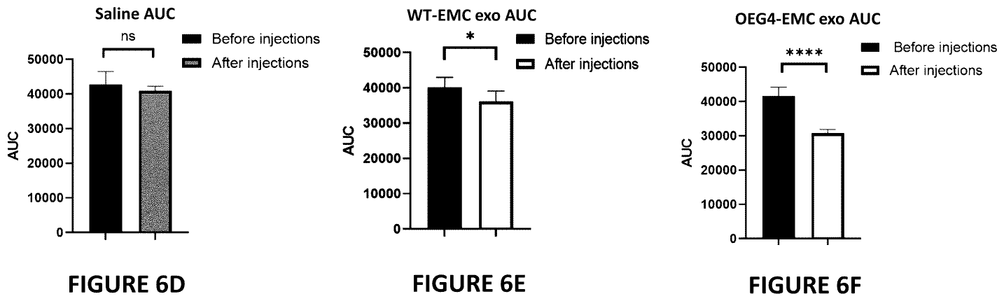

The ability of myogenic exosomes injections to improve response to glucose was tested in DIO mice model. The mice were fed with high-fat chow until diabetic phenotype in fasting glucose and glucose tolerance test (GTT) measurements, were observed. The mice were then treated with myogenic exosomes derived from 3D human muscle constructs injected 3 times separated by 3 days from each other. GTT measurements of the mice before and after injections are presented in FIGS. 6A-6B, respectively, wherein the change in glucose response of the mice injected with myogenic exosomes compared to the mice injected with saline, are clearly observed. The results were also reaffirmed quantitatively by area under the curve analysis (AUC) of the three groups after the injections (FIG. 6C). Further demonstrates is, that while mice injected with saline were not affected (FIG. 6D), injection of myogenic exosomes significantly improved glucose response (FIG. 6F). WT derived exosomes provided some effect as well, suggestibly due to the fact they are derived from healthy muscle cells, and thus, pose an effect in mice harboring a diabetic muscle tissue (FIG. 6E).

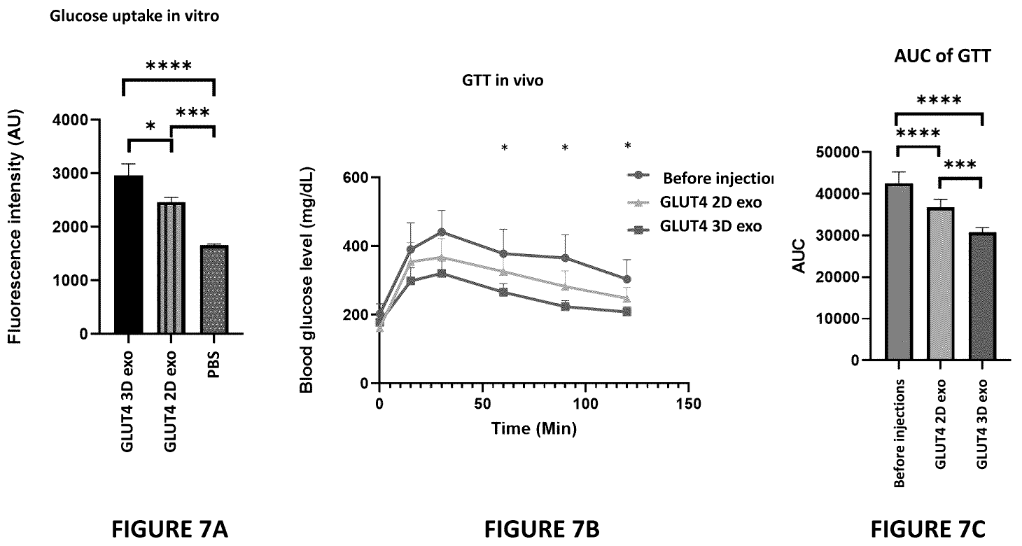

The same experiments were performed to assess the effect of exosomes derived from GLUT4 OE 3D constructs compared to exosomes derived from GLUT4 OE 2D culture. The results show that exosomes derived from GLUT4 OE 3D constructs exhibited increased effect with smaller variation compared to 2D cultures, as well as negative control (e.g., PBS), suggesting exosomes derived from GLUT4 OE 3D constructs are characterized by greater therapeutic activity and/or efficacy (FIGS. 7A-7C).

### Example 6

### Proteomic Analysis of Exosomal Content

In order to further understand the mechanisms by which myogenic exosomes can affect metabolic regulation, the inventors analyzed exosomes content using proteomic mass spectrometry (MS) analysis. Tables 1-2 highlight the substantial differences in the protein content between WT and GLUT4 OE exosomes, and suggesting that the genetic modification of the cells profoundly impact the signaling and communication the cells secrete. Interestingly, many of the proteins differentially expressed in WT and GLUT4 OE exosomes are related to regulatory pathways rather than to direct glucose uptake processes as presented in Tables 1-2.

TABLE 1
Proteins found to be increased by >50%
in OEG4 3D exosomes compared to WT 3D exosomes

| Pathway | Protein | GLUT4/WT (Fold-change) |
|---|---|---|
| Phosphoinositide 3- | COMP | 7.938560695 |
| kinases-Protein kinase B | COL1A1 | 2.252121474 |
| (PI3K-AKT) | VTN | 4.220114279 |
| | GNB2 | 1.63717 |
| | HSPAA1 | 3.110078594 |
| | YWHAZ | 2.181309876 |
| | ITGB3 | 1.700151264 |
| Advanced glycation end | COL1A1 | 2.252121474 |
| products (AGEs)-receptor | COL3A1 | 12.16588375 |
| for AGEs (RAGE) | SERPINE1 | 102.565 |
| WNT signaling | PSMA6 | 3.430478745 |
| | CLTC | 1.5478155075434 |
| | PFN1 | 1.5379 |
| Immune-response | VCL | 1.565112131 |
| regulation | C5 | 1.63783628690414 |
| | SERPINB10 | 1.67702 |
| | LRP1 | 2.540235685 |
| | C9 | 1.56620814 |
| | C6 | 1.817559188 |
| | RAB10 | 1.836899737 |
| | FTH1 | 2.180038141 |
| | PKM | 1.55868246 |
| | HSP90AA1 | 3.110078594 |
| | PPIA | 1.499942819 |

TABLE 2
Proteins found to be increased by >50%
in WT exosomes compared to OEG4 3D exosomes

| Pathway | Protein | GLUT4/WT (Fold-change) |
|---|---|---|
| Cholesterol metabolism | APOH | 0.369103724641355 |
| | APOE | 0.234974014 |
| | PLTP | 0.397077385 |
| Lipid transport | APOH | 0.369103724641355 |
| | APOE | 0.234974014 |
| | RBP4 | 0.50481932 |
| | PSAP | 0.535157 |
| | PLTP | 0.397077385 |

TABLE 3
Proteins found to be increased by >50% in
OEG4 3D exosomes compared to OEG4 2D exosomes

| Pathway | Protein | GLUT4 3D/GLUT4 2D (Fold-change) |
|---|---|---|
| AGE-RAGE | COL1A1 | 2. 2.43239 |
| | COL3A1 | 2.401433 |
| | FN1 | 2.105258 |
| Vesicle mediated | TIMP1 | 2.316251 |
| transport | VTN | 2.199595 |
| | LRP1 | 4.362978 |
| | TUBA4A | 2.309102 |
| | F2 | 2.094526 |
| | PKM | 1.615024 |
| | HSP90AA1 | 4.707336 |
| | TUBB | 6.470493 |
| | ANXA11 | 2.261048 |
| | F10 | 1.639341 |
| | DYNLL2 | 2.27433 |

For example, proteins related to immune regulation, IGF transport or cholesterol metabolism can widely affect the entire metabolic balance in the body, providing a possible explanation to the long-term effects observed in functional assays and in vivo experiments. The differences between exosomes derived from GLUT4 GE 2D vs. from 3D constructs may also be explained by increased concentration of proteins related to cytokine signaling, PI3K-AKT pathway and insulin response regulation as presented in Table 3, linking the maturity of the tissue in 3D muscle constructs to the enhanced metabolic affect and possibly more efficient communication with other tissues in vivo.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims which follow.

1. A pharmaceutical composition comprising a therapeutically effective amount of exosomes derived from cells having increased glucose transporter type 4 (GLUT4) activity.

2. The pharmaceutical composition of claim 1, wherein said cells are selected from the group consisting of: (i) cells transduced or induced to increase GLUT4 gene expression; (ii) cells induced to increase GLUT4 membrane translocation; and (iii) cells having reduced GLUT4 degradation, optionally wherein said transduced cells are transduced by a lentivirus comprising a nucleic acid sequence encoding GLUT4, and optionally wherein said nucleic acid sequence encoding said GLUT4 is operably linked to a constitutive promoter.

3.-4. (canceled)

5. The pharmaceutical composition of claim 1, wherein said cells are selected from the group consisting of: skeletal myocyte-derived cell, cardiomyocyte-derived cell, and adipocyte-derived cell or wherein said cells are selected from the group consisting of: differentiated myotube, myocyte, and myoblast.

6. (canceled)

7. The pharmaceutical composition of claim 1, wherein said cells are differentiated myotube overexpressing GLUT4.

8. The pharmaceutical composition of claim 1, wherein said exosome are derived from cells cultured in a three-dimensional (3D) scaffold or in two dimensions (2D).

9. The pharmaceutical composition of claim 1, wherein said exosome are derived from cells cultured in a 3D scaffold.

10. The pharmaceutical composition of claim 1, wherein said exosomes comprise a glucose uptake molecule, and optionally wherein said glucose uptake molecule is selected from a protein, DNA, mRNA, microRNA, long noncoding RNA, and circular RNA.

11. (canceled)

12. The pharmaceutical composition of claim 10, wherein said glucose uptake molecule is GLUT4.

13. The pharmaceutical composition of claim 1, wherein said exosomes comprise at least one protein listed under Tables 1, 2, 3, or any combination thereof, and wherein an amount of said at least one protein is modified in said exosomes compared to control exosomes, and optionally wherein said at least one protein is listed under Tables 1, 3, or both, and said amount of said at least one protein is increased in said exosomes compared to said control exosomes.

14. (canceled)

15. The pharmaceutical composition of claim 13, wherein said at least one protein is listed under Table 2, and said amount of said at least one protein is decreased in said exosomes compared to said control exosomes.

16. The pharmaceutical composition of claim 13, wherein said control comprises exosomes derived from wild-type cells or exosomes derived from cells cultured in 2D.

17. A pharmaceutical composition comprising exosomes comprising at least one protein listed under any one of Tables 1, 2, 3, and any combination thereof, wherein an amount of said at least one protein is modified in said exosomes compared to control exosomes.

18. The pharmaceutical composition of claim 17, wherein said at least one protein is listed under Tables 1, 3, or both, and said amount of said at least one protein is increased in said exosomes compared to said control exosomes, and optionally wherein increased is by at least 1.5-fold compared to control exosomes.

19. (canceled)

20. The pharmaceutical composition of claim 17, wherein said at least one protein is listed under Table 2, and said amount of said at least one protein is reduced in said exosomes compared to said control exosomes, and optionally wherein reduced amount is not more than 60% of control exosomes.

21. (canceled)

22. The pharmaceutical composition of claim 17, wherein said exosomes are derived from cells having increased GLUT4 activity.

23. The pharmaceutical composition of claim 17, wherein said control comprises exosomes derived from wild-type cells or exosomes derived from cells cultured in 2D.

24. The pharmaceutical composition of claim 1, further comprising a pharmaceutically acceptable carrier.

25. The pharmaceutical composition of claim 1, for use in treatment or prevention of diabetes mellitus or metabolic syndrome in a subject in need thereof, and optionally wherein said metabolic syndrome is selected from: obesity, pre-diabetes, and insulin resistance.

26.-27. (canceled)

28. A method for treating or preventing diabetes mellitus or metabolic syndrome in a subject in need thereof, the method comprising the steps of administering a therapeutically effective amount of the pharmaceutical composition of claim 1 to the subject, thereby treating or preventing diabetes mellitus in the subject.

29. A method for reducing glucose levels in a subject in need thereof, the method comprising the steps of administering a therapeutically effective amount of the pharmaceutical composition of claim 1 to the subject, thereby reducing glucose levels in the subject.
